(19) United States
(12) Patent Application Publication
(10) Pub. No.: US 2006/0040377 A1
(43) Pub. Date: Feb. 23, 2006

(54) Protein microarrays

(75) Inventor: Pavel Tsinberg, Carlsbad, CA (US)

Correspondence Address: FITCH EVEN TABIN AND FLANNERY, 120 SOUTH LA SALLE STREET, SUITE 1600, CHICAGO, IL, 60603-3406, US

(73) Assignee: Biocept, Inc., San Diego, CA (US)

(21) Appl. No.: 10/921,073

(22) Filed: Aug. 17, 2004

(51) Int. Cl. C12M 1/34

(52) U.S. Cl. 435/287.2

(57) ABSTRACT

Methods for making a microarray having minimal background binding of proteins by appropriately coating a substrate surface which is initially derivatized with organic functional groups. A protein-resistant polymeric coating is applied which has hydrophilic backbone polymers that are crosslinked to a substantial degree via polyfunctional isocyanate moieties. Three dimensional hydrogel microspots containing capture agents are affixed at distinct spatial locations across an array region of the surface to form a microarray. The microspots are affixed either to the substrate or to the coating. The polymeric coating preferably comprises isocyanate-capped PEG crosslinked with a polyfunctional isocyanate to form urethane polymers.

### BACKGROUND OF THE INVENTION

In the past decade or so, microarray technology has been developed as an important tool for use in a wide variety of research fields, including molecular biology, microbiology and other biological technologies. To date, the wealth of work in this area has focused on the employment of DNA arrays or those of other types of nucleic acids where a multitude of spots, i.e., microspots, are placed on a solid surface, often a glass slide or other type of “chip.” U.S. Pat. No. 5,143,854 teaches the attachment of proteins in discrete spots as an array on a glass plate and mentions a desire to expand such from proteins to create microarrays wherein cells are immobilized. This concept of creating microarrays of living cells on glass slides or other chips is also addressed in U.S. Pat. No. 6,548,263 (Apr. 15, 2003), which patent teaches the use of a glass wafer or the like which is first treated with an aminosilane to create a hydrophillic surface having reactive amino groups, a concept that is now well-known in this art. More specialized arrays have also begun to be developed for use in protein analysis which have focused both upon attaching and displaying proteins as a part of a microarray and upon analyses where DNA arrays are employed for DNA/protein interactions.

Rather than simply employing flat substrates in such protein microarrays, three-dimensional (3D) microspots have been developed using hydrogels and the like in order to better bind and present proteins as part of such a microarray. Published International Application WO02/059372 (1 Aug. 2002) shows a biochip that has been made with a plurality of microspots, in the form of optically clear hydrogel cells, attached to the top surface of the chip. These polymeric hydrogel microspots can be used either to bind proteins for interactions or to bind capture agents or probes that will subsequently react with and/or sequester proteins or peptides applied thereto in solution. For example, antigens may be bound to the surface for attachment to antibodies, or vice versa.

Background binding of proteins, carbohydrates, cell lysates and the like to the surfaces of glass or other substrates employed in microarrays, which surfaces carry microspots containing protein capture agents or the like, has posed a problem for a number of years. Non-specific binding of proteins to a microarray substrate increases the background noise when the microarray is imaged or the signals generated on the microspots are otherwise read. This makes it difficult to detect and distinguish signals being obtained from labels which should be specifically bound to particular spots, particularly in instances where a signal is relatively weak, because such background noise interferes and prevents obtaining precise readings.

To date, two of the more common methods being used to attempt to alleviate or mitigate this problem have involved manners of blocking the regions of the surface of the substrate surrounding each of the plurality of microspots. One method of blocking has chemically coated the surface of the substrate, e.g., by carrying out chemical reactions with the amino groups with which a glass surface has often been derivatized, e.g., by reacting with succinic anhydride. A second method has employed the attachment of small molecules to the glass surface, for example, BSA, tRNA, skim milk solids, casein and the like. Various of these blocking methods are described in U.S. patent Publication No. 2003/0044823, which itself proposes the use of a “spreading enhancer solution” that would presumably be effective in assays employing nucleic acid probes.

To prevent the nonspecific binding of proteins to the surface in the regions surrounding the 3D microspots and thereby reduce background signals, the above-identified International Publication suggests using monofunctional polyethylene glycol (mPEG) polymers having a reactive moiety, such as an isocyanate, at one end which will covalently bind to the amine groups, to coat regions of a surface of such a chip, or well in a plate, unoccupied by the hydrogel microspots. U.S. Pat. No. 5,672,662 (Shearwater Polymers) mentions PEG-SPA for such use in making coated substrates for use in assays involving proteins. The patent reports that methoxy-PEG-SPA (MW 5000) was grafted onto an amino-functionalized glass slide by reacting it as 5% (w/v) solution in 0.05 M sodium bicarbonate (pH 8.3) for 4 hours at 40° C. After such PEG immobilization, surfaces were rinsed with toluene, dried under vacuum and rinsed with water. It was reported that subsequent adsorption studies with fibrinogen revealed that fibrinogen adsorption on the PEG-coated surface had been substantially reduced. U.S. Pat. No. 5,932,462 to Shearwater Polymers, Inc. discloses the manufacture of a variety of such monofunctional PEGs, including branched monofunctional PEGs, and indicates that they can render surfaces nonfouling by avoiding protein adsorption, thus creating biomaterials useful in blood-related operations.

In the '263 patent, a micropatterning reaction is carried out where photo-labile or otherwise chemically removable protecting groups are first applied to the surface. Following this micropatterning, a hydrophobic substance, such as a fatty acid, is applied to react with unprotected amino groups and render these regions of the surface nonreactive with cells and proteins. Subsequently, locations in the pattern where attachment of microspots are desired are activated by removing the protecting material and applying cell adhesive material. Alternatively, it is taught that, bi-functional molecules can be applied across an entire surface containing reactive hydroxyl groups; then a mechanical stencil is used to mask areas to which it is desired that cells should later attach, while tresyl chloride-activated polyethylene glycol (PEG) is applied to react with the bi-functional molecules in the remaining regions as a cell-repulsive moiety.

It is mentioned in these patents that various labels may be used in such assay techniques to provide signals, such as fluorescence emissions, optical density or radioactivity, which need to be read or imaged. Fluorescence imaging has become more popular as these techniques have advanced, and many developments are now directed toward improving fluorescence measurements for such microarrays, either on flat plates or on multiwell plates.

It is also known that, when microarrays are created for use with fluorescent labels, there may be advantages to using a mirrored substrate in order to enhance the fluorescence signals from the labeled ligands which attach to probes carried by microspots. As set forth in U.S. Published application No. 2003/001310 (Jan. 16, 2003), a glass slide or the like may be coated with a layer of reflective metal, e.g. aluminum, and then overcoated with a layer of a dielectric material, such as silicon dioxide or alumina, which layer is, in turn, functionalized with an organic surface layer, such as an amino-modified silane. It is indicated that such coated glass slides are commercially available for use in fabricating microarrays to create arrays, and it is suggested that adapters, such as protein-binding agents, e.g. avidin, protein A, and bifunctional chemical linkers, may be used to attach capture agents. It is also taught that, after spotting the array elements onto a substrate, the remaining uncoated surface thereof should be blocked to prevent non-specific subsequent bindings, using molecules that display a hydrophilic terminus. Various blocking agents are disclosed, including, cysteine and BSA, as well as polymeric blockers, such as PEG analogs modified at at least one terminus to bind to the derivatized substrate surface, e.g., a dithiol-modified PEG having molecular weight between about 3400 and 5000. Another suggested chemical blocker is an oligomer of N-substituted glycine derivatized with hydrophilic side chains.

Although these previous attempts to solve the problem of nonspecific protein binding have shown some promise, the result has not been entirely satisfactory, and accordingly, additional solutions to this problem have been sought.

### SUMMARY OF THE INVENTION

It has now been-found that by grafting a polymer having a multifunctional hydrophilic backbone onto a substrate surface so as to coat such surface, and crosslinking such polymer to a substantial degree via linking to polyfunctional isocyanate molecules, there will be created a very effective protein-resistant microarray substrate surface. Such coatings will have improved performance in eliminating background noise, in comparison to the single-functional and bifunctional PEGs that are commercially offered for sale by Shearwater Polymers, Inc. and that have been used for this purpose.

In one particular aspect, the invention provides a microarray which comprises: a substrate having (a) a flat upper surface which is derivitized to carry organic functional groups, (b) a plurality of three-dimensional (3D) microspots at discrete spatial locations across an array region of said surface, which microspots contain or are adapted to link directly or indirectly to an organic capture agent, and (c) a protein-resistant polymeric coating covering the surface in the array region surrounding the microspots, which polymeric coating is multifunctional, comprising hydrophilic backbone polymers, which polymers are crosslinked to a substantial degree via polyfunctional isocyanate molecules, said multifunctional coating being covalently bound to said organic functional groups on said surface via isocyanate linking and providing free isocyanate groups.

In another particular aspect, the invention provides a method for making a microarray that minimizes background binding, which method comprises: providing a substrate having a flat upper surface which is derivatized with organic functional groups, applying a protein-resistant polymeric coating to cover at least an assay region of said surface by covalently binding said coating to said organic functional groups, which polymeric coating is multifunctional comprising hydrophilic backbone polymers which backbone polymers are cross-linked to a substantial degree via polyfunctional isocyanate molecules, curing said coating, affixing a plurality of three-dimensional hydrogel spots at discrete spatial locations across within said array region of said surface, and linking different organic capture agents of interest into various of said three-dimensional spots.

### DETAILED DESCRIPTION OF THE PREFERRED EMBODIMENT

The use of enzymes, antibodies, peptides, or other bioactive molecules, e.g. aptamers, has received increasing attention in creating tools for screening in the fields of bioassays and proteomics, and the use of 3-dimensional hydrogel supports for these bioactive materials in microarrays has recently gained in importance. Hydrogels are water-containing polymeric matrices. In particular, hydrogels provide a support for biomaterials that more closely resembles the native aqueous cellular environment, as opposed to a more denaturing environment that results when proteins or other such materials are directly attached to a solid support surface using some other molecular scale linkages. The present invention is believed to have particular advantage for use in the fabrication of microarrays formed with a multitude of three-dimensional (3D) microspots of hydrogel material, uniformly arranged as a matrix on a solid substrate. However, this passivation process that is herein disclosed for providing microarrays with protein-resistant regions that surround 3-dimensional microspots may also be advantageously employed with 2-dimensional microarrays, where organic probes or other moieties are affixed directly, or via short linkers, to the functionalized surface of a substrate.

The solid support or substrate employed in microarrays embodying features of the present invention may vary depending on the intended use of the product. The solid support may be any suitable material that is compatible with analytical methods in which the array is to be used, but it is preferably an impermeable, rigid material. Suitable materials include glasses, such as those formed from quartz, and silicon, as well as polymers, e.g. polyvinylchloride, polyethylene, polystyrene, polyacrylate, polycarbonate and copolymers thereof, e.g., vinyl chloride/propylene polymer, vinyl chloride/vinyl acetate polymer, styrenic copolymers, and the like. Metals and metal coatings, e.g., gold, platinum, silver, copper, aluminum, titanium and chromium and alloys thereof, may also be used.

The substrate may often be a composite of two or more different layers of material, i.e. a base as described above having one or more surface coating layers. For example, a glass base may be coated with a reflective metallic layer, e.g., gold, aluminum or titanium, overcoated first with silicon dioxide, and then functionalized with organic groups, e.g., amino-modified or thiol-modified silane, at its upper surface.

Although the solid substrate may have a variety of different configurations and dimensions depending on its intended use, a plate having at least one substantially planar surface is usually used, e.g. a slide or plate of a rectangular configuration. Commonly planar, rectangular slides are used having length and width dimensions between about 1 cm and about 40 cm; plate dimensions usually do not exceed about 30 cm and most often are about 20 cm or less. The thickness of the support will generally range from about 0.01 mm to about 10 mm, depending in part on the material from which the substrate is made so as to insure desired rigidity. The dimensions of a standard microscope slide are commonly used, i.e., about 2.54 cm. by 7.62 cm and about 1-2 mm thick.

As one example, a glass slide may be coated with a reflective aluminum layer that is over-coated with a layer of silicon dioxide or silicon monoxide having a thickness of between about 500 Å to about 2,000 Å, which thickness roughly corresponds to ¼ the wavelength of the emission or excitation light from many colorimetric labels. A layer of an aminoalkyl trialkoxysilane, such as aminopropyl triethoxysilane (APS), trichlorosilane, trimethoxysilane, or any other suitable trialkoxysilane, is coated onto the surface of the oxide; other suitable aminosilanes might also be used. The thickness of this silane layer may be from about 3 Å. to about 100 Å, more preferably about 5 Å to about 50 Å, and most preferably about 7 Å to about 20 Å. One suitable example is an APS layer that is about 7 Å thick. These amino-modified surfaces are used to directly affix the improved protein-resistant coatings and/or the 3-dimensional microspots.

Although this use of 3-dimensional microspots is preferred, binding agents for linking to organic probes or other capture moieties to be employed in the array can either be (1) attached directly to an inorganic solid surface of a substrate, or (2) attached using a functionalized top organic layer. For example, where the surface of a base, such as glass, is coated with a thin layer of a metal, such as aluminum, gold or titanium, a binding agent may be used to directly bind to a metal substrate surface without it being functionalized. For example, a thiol anchoring group may be used to bond directly to a metal, such as gold, without an intervening functionalized layer; however, a functionalized organic layer is preferably used, such as an amino-modified alkylsilane (aminosilane), as mentioned above. Where such a functionalized organic layer is used, the termini of the organic molecules of the layer provide reactive groups to which one can stably attach a binding agent or a hydrogel or the like. Suitable terminal groups are well known in this art, and such are preferably used for affixing 3-dimensional microspots to the upper surface of a substrate.

As earlier indicated, it is believed that the invention will have distinct advantages when used in the production of protein or cellular chips, particularly microarrays that utilize 3-dimensional microspots. Isocyanate-functional prepolymers for forming hydrogel microspots for such microarrays are often prepared from relatively high molecular weight polyoxyalkylene diols or polyols that are reacted with difunctional or polyfunctional isocyanate compounds. Preferred prepolymers are ones made from polyoxyalkylene diols or polyols that comprise homopolymers of ethylene oxide units or block or random copolymers containing mixtures of ethylene oxide units and propylene oxide or butylene oxide units. In the case of such block or random copolymers, at least 75% of the units are preferably ethylene oxide units. Such polyoxyalkylene diol or polyol molecular weight is preferably from about 500 to 30,000 Daltons and more preferably from about 800 to 10,000 Daltons. Suitable prepolymers may be prepared by reacting selected polyoxyalkylene diols or polyols with polyisocyanate, at an isocyanate-to-hydroxyl ratio of about 1.2 to about 2.2, so that essentially all of the hydroxyl groups are capped with polyisocyanate. Generally, polyethylene glycol (PEG), polypropylene glycol (PPG) or copolymers thereof are preferred. The isocyanate-functional prepolymers being used preferably contain active isocyanates in an amount of about 0.1 meq/g to about 2 meq/g, and more preferably about 0.2 meq/g to about 1.5 meq/g. Should relatively low molecular weight prepolymers, e.g. less than 2,000 Daltons, be used, they preferably should contain a relatively high isocyanate content (about 1 meq/g or even higher).

The inherent reactivity of prepolymers of this general type facilitates the ready covalent attachment of the polymer to a chemically functionalized substrate during polymerization. Such surfaces that are derivatized with organic functional groups are preferably provided on substrates used in fabrication of a microarray, and they facilitate affixation of polymerized hydrogel microspots in a known pattern on such a substrate, and as well as the addition of a surrounding region of a protein-resistant coating.

As mentioned above, Shearwater Polymers, Inc. markets single-functional PEGs including a variety of end-modified PEGs that may be used to couple PEGs to primary amines to render a surface nonfouling; these contain modifiers such as N-hydroxysuccinimidyl active ester (NHS), glycidyl ether (“epoxide”) and isocyanate (NCO). These modified PEGs are commercially available in a variety of sizes; for example, mPEG-succinimidyl propionate-NHS (mPEG-SPA-NHS) is sold in three sizes 2K, 5K and 20K. PEG-NHS, PEG-epoxide, PEG-NPC can be used in aqueous solvents. PEG-NCO is used in an organic solvent (NCO reacts with water) with triethylamine as a basic catalyst.

Protein-resistant polymeric coatings embodying features of the present invention may be applied to the surface of the substrate for the microarray either before or after the affixation of the three-dimensional microspots of hydrogel material. Various sequences of fabrication are described hereinafter. Organic groups of a functionalized surface are preferably used to secure the protein-resistant coating to the substrate.

When a protein is being studied in a particular assay, if there is substantial nonspecific binding, the amount of that protein that is then available for binding at a specific location on the microarray, where the complementary probe is located, is reduced; thus, the overall sensitivity of the assay is lowered. This polymeric coating effectively obviates this nonspecific binding problem for a wide range of proteins that may nonspecifically bind and create undesirable background with respect to the imaging of fluorescent signals or other colorimetric signals, (what is referred to background noise). It is well known that any decrease in the signal to background ratio hampers imaging/analysis software.

The protein-resistant polymeric coating is designed to covalently bind to the organic groups on the functionalized substrate surface; it has hydrophilic backbone polymers that are crosslinked to a substantial degree, preferably through urethane or urea bonds. Various suitable polyolefinic ether backbone polymers may be employed, including PEG, PPG, and copolymers thereof. Preferred is PEG (or a PPG copolymer thereof) which is modified with isocyanates so it will readily react with and covalently bind to organic groups on a functionalized substrate surface. As previously indicated, the organic groups attached to the surface can be any of those well known in the art, such as hydroxyl, amino, thiol or maleimide, and the derivatized hydrophilic polymer molecule is chosen accordingly to effect covalent bonding. Preferably, the PEG termini are modified with isocyanate which will covalently react with various of the usual organic groups that may be used to derivatize the substrate. The coating material is applied as a solution and allowed to react under time and other conditions suitable to crosslink and covalently bind to substantially all of the amino groups on the-functionalized surface of the substrate in the regions surrounding the microspots, which is referred herein as curing. The coating can alternatively be applied across the entire array region, or in a pattern surrounding locations where microspots are to be located prior to the affixation of 3D microspots. These isocyanate-modified molecules create a strong urea bond with amino groups on a surface that has been derivatized with an aminosilane or the like.

Where PEG backbone polymers are used in the coating, any suitable organic polyisocyanate, such as an aliphatic, alicyclic, araliphatic, or aromatic polyisocyanate, may be used to devivative these molecules, either singly or in mixtures of two or more; aromatic and aliphatic isocyanates are preferred. Aromatic isocyanate compounds are generally more economical and reactive with hydroxyls than are aliphatic isocyanate compounds, and they are often the more preferred. Suitable aromatic isocyanate compounds include: 2,4-toluene diisocyanate (TDI), 2,6-toluene (present in commercial TDI) diisocyanate, an adduct of TDI with trimethylolpropane (available as DESMODUR CB from Bayer Corporation, Pittsburgh, Pa.), the isocyanurate trimer of TDI (available as DESMODUR IL from Bayer), diphenylmethane 4,4′-diisocyanate (MDI), diphenylmethane 2,4′-diisocyanate, 1,5-diisocyanato-naphthalene, 1,4-phenylene diisocyanate, 1,3-phenylene diisocyanate, 1-methyoxy-2,4-phenylene diisocyanate, 1-chlorophenyl-2,4-diisocyanate, and mixtures thereof. Among the aromatic isocyanates, particularly preferred are TDI and MDI.

Examples of useful alicyclic isocyanate compounds include the following: dicyclohexylmethane diisocyanate (commercially available as DESMODUR W, available from Bayer), 4,4′-isopropyl-bis(cyclohexylisocyanate), isophorone diisocyanate (IPDI), cyclobutane-1,3-diisocyanate, cyclohexane 1,3-diisocyanate, cyclohexane 1,4-diisocyanate (CHDI), 1,4-cyclohexanebis(methylene isocyanate) (BDI), 1,3-bis(isocyanatomethyl)cyclohexane 3-isocyanatomethyl-3,5,5-trimethylcyclohexyl isocyanate, and mixtures thereof.

Examples of useful aliphatic isocyanate compounds include: 1,4-tetramethylene diisocyanate, hexamethylene 1,4-diisocyanate, hexamethylene 1,6-diisocyanate (HDI), 1,1,2-dodecane diisocyanate, 2,2,4-trimethyl-hexamethylene diisocyanate or 2,4,4-trimethyl-hexamethylene diisocyanate (TMDI), 2-methyl-1,5-pentamethylene diisocyanate, dimer diisocyanate, the urea of hexamethylene diisocyanate, the biuret of hexamethylene 1,6-diisocyanate (HDI) (available as DESMODUR-100 and -3200 from Bayer), the isocyanurate of HDI (available as DESMODUR-3300 and -3600 from Bayer), a blend of the isocyanurate of HDI and the uretdione of HDI (available as DESMODUR-3400 from Bayer), and mixtures thereof.

Examples of useful araliphatic include of m-tetramethyl xylylene diisocyanate (m-TMXDI), p-tetramethyl xylylene diisocyanate (p-TMXDI), 1,4-xylylene diisocyanate (XDI), 1,3-xylylene diisocyanate, p-(1-isocyanatoethyl)-phenyl isocyanate, m-(3-isocyanatobutyl)-phenyl isocyanate, 4-(2-isocyanatocyclohexylmethyl)-phenyl isocyanate, and mixtures thereof.

For purposes of this application, polyfunctional is intended to mean 3 or more functional groups. Suitable triisocyanates can be obtained by reacting three moles of a diisocyanate with one mole of a triol. For example, toluene diisocyanate, 3-isocyanatomethyl-3,4,4-trimethylcyclohexyl isocyanate, or m-tetramethylxylene diisocyanate can be reacted with 1,1,1-tris(hydroxymethyl)propane to form triisocyanates. Such a product from the reaction with m-tetramethylxylene diisocyanate is commercially available as CYTHANE 3160 (American Cyanamid, Stamford, Conn.).

As earlier indicated, it is desired that the polymeric protein-resistant coatings which are applied are crosslinked to a substantial degree. By crosslinking to a substantial degree is meant that crosslinks are created between the backbone polymers at at least about 2.5% and preferrably at least about 5% of the polyisocyate molecules (which are thus linked to at least 3 different backbone polymers); more preferably at least about 10%, and most preferably at least about 20% of the polyfunctional molecules have such triple linkages. When the backbone molecules are polyoxyalkylene diols or polyols or other such polyethers, they may be applied as polyurethane prepolymers where they are derivatized by difunctional or trifuncitional isocyantes as heretofore described. Their crosslinking or curing can be completed simultaneously with the covalent bonding to the organic moieties attached to the surface of the substrate, as by applying such a prepolymer as part of an aqueous solution; alternatively, the crosslinking reaction can be catalyzed as well known in the art.

The polymeric coating that is applied has no truly significant thickness, as it may be essentially a monomolecular layer. Usually, it will be at least about 3 molecular layers thick, and generally the thickness will not be greater than about 0.1 micron. However, in those instances where the entire array region of a substrate is first coated with the protein-resistant coating, the characteristics of the coating are selected and regulated so as to provide sufficient reactive groups to which the 3D microspots can subsequently strongly bind. In this respect, when the microspots are hydrogels formed from isocyanate-capped polyurethanes, a protein-resistant coating wherein about 50% or more of the polyisocyanate molecules have at least 1 unreacted isocyanate moiety provides sufficient platforms for the subsequent affixation of such 3D microspots.

Overall, the invention provides various sequences or procedures for carrying out the fabrication of microarrays having these protein-resistant surfaces. As one embodiment of such microarrays, commercially available glass slides are employed that have a reflective aluminum layer that is overcoated with a layer of silicon dioxide, which is in turn coated with an aminosilane to provide functionalized amino groups. Slides such as these are commercially available from Erie Scientific Company and from TeleChem International, Inc.

The following examples illustrate several applications relating to protein chips. It should of course be appreciated that these examples of antigen-antibody interactions (and other such interactions mentioned herein) are only illustrative of working examples and do not constitute limitations upon the invention which is defined in the appended claims.

### EXAMPLE 1

### Coating Applied when Microspots are Already in Place to Block Non-Specific Binding and Lower Background Noise of Slide

This experiment employs a hydrogel platform as a matrix for anchoring antibodies therewithin. Antibody-antigen interactions are routinely employed in a variety of biological assays, and the ability to anchor either component (antibody or antigen) is a desirable feature for a substrate antigen is a desirable feature for a substrate to create such a microarray. With the microspots containing desired capture agents in place, the polymeric protein-resistant coating is applied.

A trehalose stock solution, 50% w/v D(+) trehalose dihydrate in 50 mM sodium borate aqueous buffer, pH 8.0, is added to 50 μl final volume hydrogel formulation. The formulation includes 3.5 weight % final concentration HYPOL PreMA® G-50 hydrogel prepolymer (premixed stock solution containing HYPOL, acetonitrile, N-methyl-2-pyrrolidinone at a w/w/w ration of 1:3:3, respectively), anti-transferrin (4 mg/ml phosphate buffered saline 1× (PBS), 2 μl bovine 1 gG (50/mg/ml in PBS and 1.25% glycerol). Trehalose is included to provide a final w/v percentage of about 5% trehalose. Blank 3D hydrogel spots which do not contain protein are included.

Multiple microdroplets of the test solutions are spotted using multiple pins onto an aminosilane-coated glass slide along with mulitple microdroplets of blank hydrogel. The test protein being encapsulated is anti-transferrin, and the hydrogel formulation is allowed to fully cure for at least about 180 minutes at about 19° C. in 94 to 95% RH.

A solution containing 0.05% of MDI dervitized PEG triol is prepared in an appropriate solvent i.e. acetonitrile. The molecular weight of the PEG backbone is about 10,000 molecular weight units (Daltons). To a solution of 20 ml of acetonitrile/PEG, 20 μL of triethylamine (TEA) is added as a basic catalyst. Without allowing protein hydrogel microspots to dry, the slide is dipped into the PEG/acetonitrile/TEA solution for about 10 seconds, it is then rinsed for 10 seconds in clean acetonitrile, followed by an aqueous rinse in 1× PBS at pH 7.4.

The system is incubated with Cy3 fluorescent dye-labeled transferrin (Amersham, approximately 0.1 μg/ml in PBS containing 0.1% Triton X100 (PBST), and 1% bovine serum albumin (BSA)) at 45° C. with shaking periodically. Following incubation, the slide is washed 2×10 minutes in PBST and then imaged using a ScanArray Lite slide scanner. The blank hydrogel spots show no detectable signal, and the trehalose-antibody spots have a strong signal. The Cy3-labeled transferrin specifically binds to its natural ligand within the hydrogel microspots, and there is little detectable binding activity to either the hydrogel itself, or to the glass substrate. The absence of significant signal from the regions of the slide surrounding the microspots shows the effectiveness of this coating in preventing nonspecifically bound proteins from binding to the substrate while not interfering with the achievement of complexes within the 3D microspots.

### EXAMPLE 2

### Use of Pre-Applied Coating

As noted in the previous example, antibody-antigen reactions are routinely employed in biological assays. In this example, the coating is pre-applied, and as opposed to anchoring the antibody, an antigen is anchored within the 3D hydrogel matrix.

A solution of 1% MDI derivitized PEG triol is prepared in an appropriate solvent, i.e., acetonitrile. Repel-Silane ES is added as a hydrophobic agent to lessen the hydrophilic effect of PEG. Repel-Silane is a 2% solution of dimethyldichlorosilane dissolved in octamethyl cyclo-octasilane. The molecular weight of the PEG backbone is about 10,000 molecular weight units. To a solution of 20 ml of acetonitrile/PEG, 20 μL of triethylamine (TEA) is added as a basic catalyst. Slides are incubated in PEG/acetonitrile/TEA solution for 10 minutes at room temperature with agitation. They are then washed in clean acetonitrile 3× for 10 minutes with agitation. Following the last acetonitrile wash, slides are washed in DI water for 1 hour, then rinsed in ethanol, and dried.

Using the methodology described in Example 1, the protein antigen, human transferrin (0.2 mg/ml), is directly immobilized at different dilutions in 3.3% hydrogel with 5% trehalose, 2 mg/ml BSA onto such a pre-treated glass slide as a plurality of 3D microspots. The slide is incubated for 1 hour with mouse ascites fluid containing anti-human transferrin at the varying concentrations. After incubation, the slide is washed three times for 10 minutes with PBST. The bound, mouse, anti-transferrin antibody is visualized by incubating the slide with Cy3-labeled donkey anti-mouse IgG, followed by laser scanner imaging. A linear dose response is observed over three orders of magnitude of dilutions, i.e. 0.1 to 0.001, which indicates the functionality of the antigen anchored within the hydrogel matrix and the permeability of the hydrogel matrix supporting sequential diffusion of antibodies into the matrix as part of the overall assay methodology.

The Cy3-labeled secondary antibody demonstrates that the primary anti-transferrin antibody specifically binds to its natural ligand within the hydrogel microspots, and there is little detectable binding activity to either the hydrogel itself or to the coated glass substrate. The absence of significant signal from regions of the slide surrounding the 3D microspots indicates the coating is effective in preventing nonspecifically bound proteins from binding to the substrate without interfering with the achievement of complexes within the 3D microspots.

Although the invention has been described with respect to a number of different embodiments which include the best modes presently contemplated by the inventor, it should be understood that changes and modifications as would be obvious to one skilled in this art is set forth in the claims appended hereto. For example, although there are advantages in the use of biochips having a plurality of microspots carrying different capture agents, in certain situations biochips carrying only one capture agent may be desired. The disclosures of all patents and publications set forth hereinbefore are incorporated herein by reference.

Particular features of the invention are emphasized in the claims which follow.

1. A microarray which comprises:
a substrate having a flat upper surface which is derivitized to carry organic functional groups,
a plurality of three-dimensional (3D) microspots at discrete spatial locations across an array region of said surface, which microspots contain or are adapted to link directly or indirectly to an organic capture agent, and
a protein-resistant polymeric coating covering the surface in the array region surrounding the microspots, which polymeric coating is multifunctional, comprising hydrophilic backbone polymers, which polymers are crosslinked to a substantial degree via polyfunctional isocyanate molecules, said multifunctional coating being covalently bound to said organic functional groups on said surface via isocyanate linking and providing free isocyanate groups.

2. The microarray of claim 1 wherein said hydrophilic backbone polymer is a polyolefinic ether.

3. The microarray of claim 2 wherein said polymer is a polyolefinic ether polyol that is end-capped with isocyanate groups through urethane linkages.

4. The microarray of claim 3 wherein said polyolefinic ether polyol is a polyethylene glycol (PEG), a polypropylene glycol (PPG) or a copolymer thereof.

5. The microarray of claim 4 wherein said polyol is PEG, PPG or copolymer thereof having a molecular weight between about 500 and 30,000 Daltons.

6. The microarray in accordance with claim 3 wherein said end-capped polyolefinic ether polymer is cross-linked by urea bonds to said polyfunctional isocyanate moieties.

7. The microarray according to claim 6 wherein said backbone polymers are crosslinked through aromatic or aliphatic polyfunctional isocyanate molecules.

8. The microarray according to claim 7 wherein at least about 2.5% of said polyfunctional isocyanate molecules present have each of their functional groups linked to separate backbone polymers to effect said cross linking to a substantial degree.

9. The microarray according to claim 1 wherein said organic functional groups which derivitize said substrate are amine moieties.

10. The microarray according to claim 1 wherein said substrate is a glass slide, the upper surface of which is derivatized by an aminosilane.

11. The microarray according to claim 1 wherein said 3D spots are polyurethane-based hydrogels.

12. The microarray according to claim 11 wherein said hydrogel spots are bound directly to said substrate via said organic functional groups.

13. The microarray according to claim 11 wherein said hydrogel spots are affixed via said free isocyanate groups to said polymeric coating which is bound to said substrate across the array region.

14. A method for making a microarray that minimizes background binding, which method comprises:
providing a substrate having a flat upper surface which is derivatized with organic functional groups,
applying a protein-resistant polymeric coating to cover at least an assay region of said surface by covalently binding said coating to said organic functional groups, which polymeric coating is multifunctional comprising hydrophilic backbone polymers which backbone polymers are cross-linked to a substantial degree via polyfunctional isocyanate molecules,
curing said coating,
affixing a plurality of three-dimensional hydrogel spots at discrete spatial locations across within said array region of said surface, and
linking different organic capture agents of interest into various of said three-dimensional spots.

15. The method according to claim 14 wherein said linking of said organic capture agents is effected after affixing said three-dimensional spots to said surface.

16. The method according to claim 14 wherein said hydrogel spots are bound directly to said substrate via said organic functional groups prior to applying said coating.

17. The method according to claim 14 wherein said hydrogel spots are affixed to said polymeric coating after said coating is bound to said substrate across the array region and said spots are bound to said coating.

18. The method according to claim 14 wherein prior to application of said protein-resistant coating, said upper surface is patterned with protective material to cover regions where three-dimensional spots will subsequently be located, and wherein said protective material is subsequently removed after said protein-resistant coating is in place to permit the affixation of said three-dimensional spots.

19. The method according to claim 14 wherein said polymeric coating comprises backbone polymers of polyethylene glycol (PEG), polypropylene glycol (PPG) or copolymers thereof that is end-capped with isocyanate groups through urethane linkages which backbone polymers are cross-linked by urea bonds to said polyfunctional isocyanate molecules.

20. The method according to claim 14 wherein said organic capture agents contained in said hydrogel spots when such are affixed.
